(19) United States
(12) Patent Application Publication
(10) Pub. No.: US 2014/0314697 A1
(43) Pub. Date: Oct. 23, 2014

(54) Method for Inhibiting Inflammation and Reducing Melanophilin Expression with Glycine Derivatives And the Composition Thereof

(71) Applicant: CORUM INC., Taipei City (TW)

(72) Inventors: Ssu-Ching Wang, Taipei City (TW); Pei-Hsuan Wu, Taipei City (TW); Jun-Yi Yao, Taipei City (TW); Nai-Hsuan Hsu, Taipei City (TW)

(73) Assignee: CORUM INC., Taipei City (TW)

(21) Appl. No.: 14/255,114

(22) Filed: Apr. 17, 2014

Related U.S. Application Data
(60) Provisional application No. 61/813,251, filed on Apr. 18, 2013.

(51) Int. Cl. C07C 237/06; A61Q 19/02; A61K 8/42

(52) U.S. Cl. CPC C07C 237/06; A61K 8/42; A61Q 19/02
USPC 424/62; 514/563

(57) ABSTRACT

This invention discloses method for inhibiting inflammation and reducing melanophilin expression with glycine derivatives and the composition thereof. The mentioned glycine derivatives can not only efficiently inhibit skin inflammation, but also can present significant melanophilin expression reduction. This mentioned composition of this invention comprises the glycine derivatives, and shows melanophilin expression reduction and anti-inflammatory effect. The mentioned composition can be applied in topical cosmetic or pharmaceutical compositions as skin care preparations, or functional preparations.

### BACKGROUND OF THE INVENTION

1. Field of the Invention

The present invention is generally related to glycine derivatives, and more particularly to method for inhibiting inflammation and for reducing melanophilin expression with glycine derivatives and composition thereof.

2. Description of the Prior Art

The skin can be structurally classified into three main layers, including epidermis, dermis and subcutis. Each layer has its specific structure and functions. Epidermis is seen on the surface of the skin, containing large numbers of keratinocytes. As the outermost layer of skin, the epidermal keratinocytes would be the major target of UV irradiation.

Moderate UV exposure can induce the production of cutaneous vitamin D, which is a nutrient for bone heath. But excessive UV exposure may result in acute and chronic health effects on the skin, eye and immune system. Sunburn (erythema) is the best-known acute inflammatory effect on skin of excessive UV irradiation with dermal vasodilatation and leukocyte infiltration, and chronic inflammation on skin is generally relative to aging.

Due to UV stimulation, keratinocytes will act as iniciators of inflammation by producing and releasing pro-inflammatory mediators like IL-1, IL-6, IL-8, TNF-α, PGE2. IL-1, IL-6 and TNF-α are involved in both acute and chronic inflammatory responses, and IL-8 is an acute inflammatory chemokine that can recruit more pro-inflammatory cells.

Besides, Melanophilin, Mlph, is a protein that involved in the transport of melanosomes. These lysosome-related organelles are specialized in the synthesis and distribution of melanin. Mlph is an essential member of the melanosome trafficking complex, acting as a link between Rab27a and myosin Va. It may also be involved in the trafficking of epithelial Na+ channel in cells of the collecting duct of the kidney. Besides, there are some evidences show a positive relationship between estrogen receptor positive breast carcinoma and MLPH gene expression.

In view of the above matter, developing a novel method and composition for inhibiting inflammation and reducing melanophilin expression with glycine derivatives is an important task for the industry.

### SUMMARY OF THE INVENTION

In light of the above background, in order to fulfill the requirements of the industry, the present invention provides a novel method and composition for inhibiting inflammation and reducing melanophilin expression with glycine derivatives having the adventures of high safety to human body, so that the glycine derivatives can be applied in various topical compositions.

One object of the present invention is to provide a method for inhibiting topical inflammation with glycine derivatives.

Another object of the present invention is to provide a method for reducing melanophilin expression with glycine derivatives on transcription level.

Still another object of the present invention is to provide a method for reducing melanophilin expression with glycine derivatives on translation level.

Still another object of the present invention is to provide a composition comprising glycine derivatives to provide melanophilin expression reduction and anti-inflammatory effect, and be able to be applied in topical cosmetic or pharmaceutical compositions as skin care preparations, or functional preparations.

Accordingly, the present invention discloses method for inhibiting inflammation and reducing melanophilin expression with glycine derivatives and the composition thereof. The general formula of the mentioned glycine derivatives is as the following.

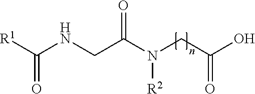

The mentioned glycine derivatives shows not only melanophilin expression reduction but also anti-inflammatory effect.

This invention also discloses a composition for inhibiting inflammation and reducing melanophilin expression. The mentioned composition comprises the mentioned glycine derivatives with addition amount of 0.05-10 wt % (weight ratio). The composition can be applied in skin care preparations, such as facial and/or body cleansers, toner, moisturizing spray, face masks, serum, day cream, night cream, eye cream, feminine halo cream, body cream, hand cream, hand wash, body wash, feminine hygiene cleanser. Preferably, the mentioned composition of this invention can be applied in topical cosmetic or pharmaceutical compositions as skin care preparations, or functional preparations.

### BRIEF DESCRIPTION OF THE DRAWINGS

The present disclosure can be described by the embodiments given below. It is understood, however, that the embodiments below are not necessarily limitations to the present disclosure, but are used to a typical implementation of the invention.

FIG. 1 shows the inhibitory effect 0.5% Ac-Gly-β-Ala shows on Mlph in transcriptional (mRNA) level;

FIG. 2 shows the inhibitory effect of Ac-Gly-β-Ala on Mlph in translational (protein) level with dose-dependence;

FIG. 3 shows the dose-dependent inhibitory effect of 0.05-0.4% Ac-Gly-β-Ala on Mouse IL-6 secretion;

FIG. 4 shows the dose-dependent inhibitory effect of 0.05-0.4% Ac-Gly-β-Ala on Mouse MIP-2 secretion;

FIG. 5 shows the good cell viability of 0.05-0.4% Ac-Gly-β-Ala on Raw264.7;

FIG. 6 shows the dose-dependent inhibitory effect of 0.05-0.4% Ac-Gly-β-Ala on Human IL-6 secretion;

FIG. 7 shows the dose-dependent inhibitory effect of 0.05-0.4% Ac-Gly-β-Ala on Human IL-8 secretion;

FIG. 8 shows the good cell viability of 0.05-0.4% Ac-Gly-β-Ala on NHEK;

FIG. 9 shows the inhibitory effect of 0.2% Glycine derivatives similar to IL-6 inhibitory effect with 0.2% Ac-Gly-β-Ala (15% IL-6 production), wherein cell viability is higher than 90% in all of dosing solution; and

FIG. 10 shows the inhibitory effect 0.2% Glycine derivatives similar to IL-8 inhibitory effect with 0.2% Ac-Gly-β-Ala (69% IL-8 production), wherein cell viability is higher than 90% in all of dosing solution.

### DESCRIPTION OF THE PREFERRED EMBODIMENTS

What probed into the invention is a method for inhibiting inflammation and reducing melanophilin expression with glycine derivatives and the composition thereof. Detailed descriptions of the structure and elements will be provided in the following in order to make the invention thoroughly understood. Obviously, the application of the invention is not confined to specific details familiar to those who are skilled in the art. On the other hand, the common structures and elements that are known to everyone are not described in details to avoid unnecessary limits of the invention. Some preferred embodiments of the present invention will now be described in greater details in the following. However, it should be recognized that the present invention can be practiced in a wide range of other embodiments besides those explicitly described, that is, this invention can also be applied extensively to other embodiments, and the scope of the present invention is expressly not limited except as specified in the accompanying claims.

One preferred embodiment according to this specification discloses a method for reducing melanophilin expression. The mentioned method comprises a step for applying glycine derivatives to a target region. In one preferred example of this embodiment, the target region can be cells. In another preferred example, the target region can be skin of a mammal. The general formula of the mentioned glycine derivatives is as the following.

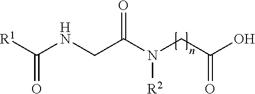

In the above-mentioned general formula, R¹ represents a C₁-C₄ alkyl group, R² represents a hydrogen atom or a methyl group, and n represents an integer of 1-6.

In one preferred example of this embodiment, the mentioned glycerin derivatives is 3-(2-acetylamino-acetylamino)-propionic acid, or named as Acetyl-Glycine-β-Alanine (hereinafter abbreviated as Ac-Gly-β-Ala), shown as the following structure.

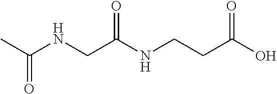

In another preferred example of this embodiment, the mentioned glycerin derivatives is 4-(2-Acetylamino-acetylamino)-butyric acid, or named as Acetyl-Glycine-γ-aminobutyric acid (hereinafter abbreviated as Ac-Gly-GABA), shown as the following structure.

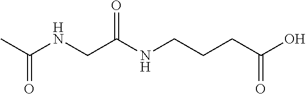

In still another preferred example of this embodiment, the mentioned glycerin derivatives is [(2-Acetylamino-acetyl)-methyl-amino]-acetic acid, or named as Acetyl-Glycine-Sarcosine (hereinafter abbreviated as Ac-Gly-Sar), shown as the following structure.

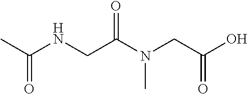

Another preferred embodiment according to this specification discloses a method for inhibiting inflammation with glycine derivatives. The mentioned method comprises a step for applying glycine derivatives to a target region. In one preferred example of this embodiment, the target region can be cells. In another preferred example, the target region can be skin of a mammal. The general formula of the mentioned glycine derivatives is as the following.

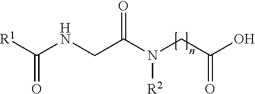

In the above-mentioned general formula, R¹ represents a C₁-C₄ alkyl group, R² represents a hydrogen atom or a methyl group, and n represents an integer of 1-6.

In one preferred example of this embodiment, the mentioned glycerin derivatives is 3-(2-acetylamino-acetylamino)-propionic acid, or named as Acetyl-Glycine-β-Alanine (hereinafter abbreviated as Ac-Gly-β-Ala), shown as the following structure.

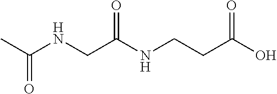

In another preferred example of this embodiment, the mentioned glycerin derivatives is 4-(2-Acetylamino-acetylamino)-butyric acid, or named as Acetyl-Glycine-γ-aminobutyric acid (hereinafter abbreviated as Ac-Gly-GABA), shown as the following structure.

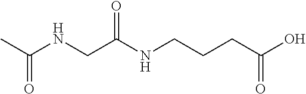

In still another preferred example of this embodiment, the mentioned glycerin derivatives is [(2-Acetylamino-acetyl)-methyl-amino]-acetic acid, or named as Acetyl-Glycine-Sarcosine (hereinafter abbreviated as Ac-Gly-Sar), shown as the following structure.

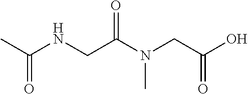

The preferred examples of the method for inhibiting inflammation and reducing melanophilin expression with glycine derivatives and the composition thereof according to the invention are described in the following. However, the scope of the invention should be based on the claims, but is not restricted by the following examples.

### Example 1

### Reduction of Melanophilin Expression on Transcriptional Level: (Hereinafter Ac-Gly-β-Ala is Used for Reducing Melanophilin Expression, and this Specification is not Limited by the Following Examples)

1.1. Materials:

Part A.: Murine melanoma cell

Murine melanoma cell line B16-F10 (BCRC No. 60031)

Rinse Buffer: Sterile D-PBS

Complete Medium: DMEM with 10% Fetal Bovine Serum

Quality Control of qPCR primer:

| Item | Brand | Cat No |
|---|---|---|
| Mlph (melanophilin) | GeneCopoeia | MQP036880 |

1.2. General Procedure:

a. Rinse the confluent B16-F10 cells with sterile D-PBS then trypsinize all cells and perform cell counting.

b. Seeding 2E+06 cells per 10-cm dish.

c. Incubate B16F10 cells at 37° C., 5% CO₂ for about 24 hours.

d. Prepared 0.5% testing material in Complete Medium.

e. Discard the medium and add 9 mL Complete Medium and 0.5% testing material into B16-F10 cells in 10-cm dish.

f. Incubate B16-F10 cells at 37° C., 5% CO₂ for about 1 hour.

g. After incubating for about 1 hour, discard the supernatant and wash the B16-F10 cells with sterile D-PBS.

h. Add 3 mL Trizol to suspend cells and qPCR test by Welgene Biotech Co., Ltd (outsourcing test).

2.3. Data Analysis:

$$Percentage to Control ( \% ) = \frac{0.5 \% testing materials}{Negative Control} \times 100 \%$$

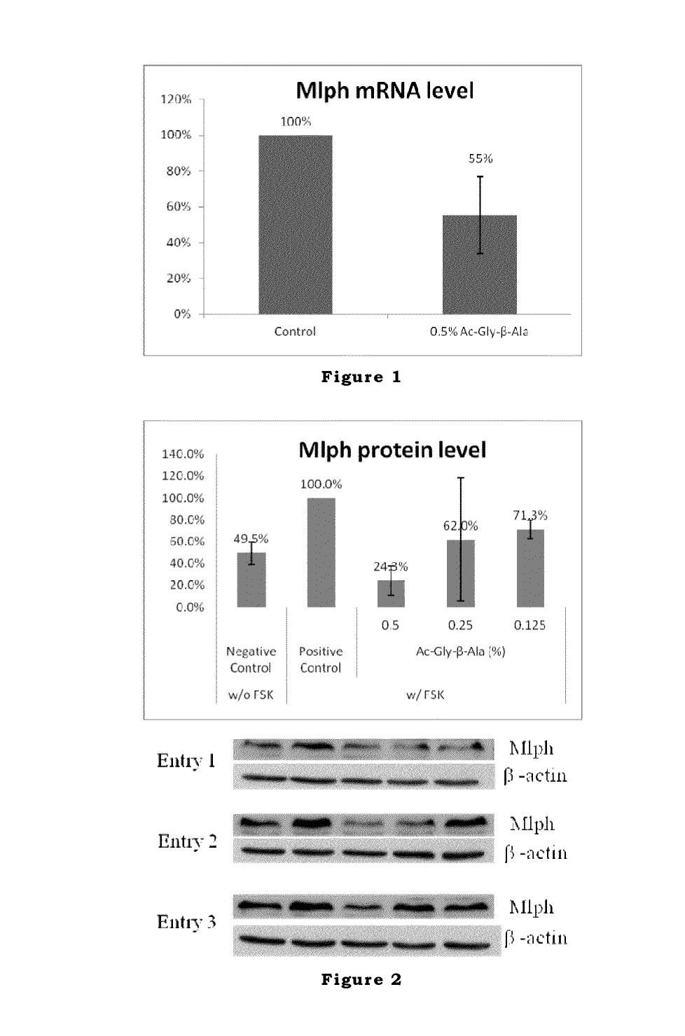

Referred to FIG. 1, 0.5% Ac-Gly-β-Ala shows inhibitory effect onMlph in transcriptional (mRNA) level. And, the experimental data of Mlph mRNA expression level is as the Table 1.

TABLE 1
Data Analysis of Mlph mRNA expression
level inhibited by 0.5% Ac-Gly-β-Ala

| | Entry | | | | |
|---|---|---|---|---|---|
| | Entry 1 | Entry 2 | Entry 3 | Average | Stdev |
| Control | 100% | 100% | 100% | 100% | 0% |
| 0.5% | 80% | 40% | 46% | 55% | 22% |
| Ac-Gly-β-Ala | | | | | |

### Example 2

### Reduction of Melanophilin Expression on Translational Level (Hereinafter Ac-Gly-β-Ala is Used for Reducing Melanophilin Expression, and this Specification is not Limited by the Following Examples)

2.1. Materials:

Part A.: Murine melanoma cell

Murine melanoma cell line B16-F10 (BCRC No. 60031)

Rinse Buffer: Sterile D-PBS

Complete Medium (as negative control): DMEM with 10% Fetal Bovine Serum

Testing Medium (as positive control): 20 μM forskolin (FSK) in Complete Medium

Antibodies:

| Item | Brand | Cat No |
|---|---|---|
| Primary Ab: Mlph | Santa Cruz | Sc-33800 |
| (melanophilin) | | |
| Sencodary Ab: anti-rabbit | Jackson | 111-035-003 |

2.2. General Procedure:

a. Rinse the confluent B16-F10 cells with sterile D-PBS then trypsinize all cells and perform cell counting.

b. Seeding 2.5E+05 cells per 6-cm dish.

c. Incubate B16F10 cells at 37° C., 5% CO₂ for about 24 hours.

d. Prepared 0.5%/0.25%/0.125% testing material in Testing Medium.

e. Discard the medium and add 3 mL controls and testing material into B16-F10 cells in 6-cm dish.

f. Incubate B16-F10 cells at 37° C., 5% CO₂ for about 48 hour.

g. After incubating for about 48 hours, discard the supernatant and wash the B16-F10 cells with sterile D-PBS.

h. Mlph protein expression is measured by western blot. Western blot conditions:
Loading protein amount: 40 μg/well
Protein transfer by Semi-dry Transfer Apparatus
Immunoblot with Primary Ab (1:250 dilution) at 4° C. overnight
Incubate with secondary Ab (1:5000 dilution) at room temperature for 1 hour
Detection was performed with the ECL system
Relative quantification of protein expression was measured by IMAGEJ free software). β-actin was used as the internal control for normalization
Data Analysis:

$$Percentage to positive control ( \% ) = \frac{Mlph / β - {actin}_{testing material}}{Mlph / β - {actin}_{positive control}} \times 100 \%$$

Referred to FIG. 2, Ac-Gly-β-Ala shows inhibitory effect on Mlph in translational (protein) level. The experimental data of Mlph protein expression level is as the Table 2.

TABLE 2
Data Analysis of Mlph protein expression level
inhibited by Ac-Gly-β-Ala with dose-dependence

| | | | Entry | | | Aver- | |
|---|---|---|---|---|---|---|---|
| | | | Entry 1 | Entry 2 | Entry 3 | age | Stdev |
| w/o | Negative | | 42.9% | 43.9% | 61.7% | 49.5% | 10.6% |
| FSK | Control | | | | | | |
| w/FSK | Positive | | 100.0% | 100.0% | 100.0% | 100.0% | 0.0% |
| | Control | | | | | | |
| | Ac-Gly- | 0.5 | 36.5% | 10.6% | 25.8% | 24.3% | 13.0% |
| | β-Ala | 0.25 | 49.4% | 12.8% | 123.8% | 62.0% | 56.6% |
| | (%) | 0.125 | 66.8% | 65.7% | 81.2% | 71.3% | 8.7% |

### Example 3

### Skin Whitening Test: (Hereinafter Ac-Gly-β-Ala is Used for Skin Whitening Test, and this Specification is not Limited by the Following Examples)

In this example, the in-vivo skin test is finished by AMA Laboratories USA. The skin color changes are evaluated with Minolta chromameter color computer system. The Age spot photography is evaluated with Reverse Photo Engineering Quantitative Analysis.

In terms of skin color changes, the participants were women aged 25-48 years old, wherein nine of the participants were caucasian, and one was Asian. The participants were tested before (baseline), and after using 14, 28, 56 days with Minolta chromameter color computer system to perform the skin color change evaluation. And the samples used for the skin color change evaluation is 0.2% Ac-Gly-β-Ala. The test results of the kin color changes (L* value) are presented in the following Table 3A.

TABLE 3A
Hyper pigmentation Reduction - Skin Color Data

| | Day 14 | Day 28 | Day 56 |
|---|---|---|---|
| % Difference: | 26.90%* | 41.07%* | 41.64%* |
| Max % Improvement: | 62.84% | 87.34% | 87.97% |

*Statistically Significant

In terms of age spot photography, the participants were 5 women aged 38-49 years old, wherein four of the participants were caucasian, and one was Asian. The participants were tested before (baseline), and after using 14, 28, 56 days with high-resolution digital camera, then Reverse Photo Engineering quantitative analysis to perform the age spot change evaluation.

The test results of the Age spot changes are presented in the following Table 3B.

TABLE 3B
Age Spot Reduction - Reverse Photo Engineering Data

| | Day 14 | Day 28 | Day 56 |
|---|---|---|---|
| % Difference: | −41.47%* | −63.37% | −73.07%* |
| Max % Improvement: | −51.67% | −80.63% | −87.40% |

*Statistically Significant

According to the above test results of this example, it is obvious that the test sample with 0.2% Ac-Gly-β-Ala can increase the L* value of skin. That is, the sample containing 0.2% Ac-Gly-β-Ala is efficiently on skin whitening. Besides, according to the Age spot evaluated with image system, after using the sample containing 0.2% Ac-Gly-β-Ala, the quantitative analyzed data for evaluating the age spot change evaluation is significantly decreased. Therefore, this example provides the evidence that Ac-Gly-β-Ala is with skin whitening performance.

### Example 4

### Anti-Inflammation: (Hereinafter Ac-Gly-β-Ala is Used for Anti-Inflammation, and this Specification is not Limited by the Following Examples)

4.1. Materials:

Part A.: murine macrophage cell

Murine macrophage cell line RAW264.7 (BCRC No. 60001)

Rinse Buffer: Sterile D-PBS

Complete Medium (as negative control): DMEM medium with 10% Fetal Bovine Serum

Testing Medium (as positive control): 100 ng/mL LPS and 10 ng/mL IFN-r in Complete Medium

Test kits: (ELISA kit)

| Item | Brand | Cat. No |
|---|---|---|
| Mouse IL-6 Quantikine ELISA kit | R&D | M6000B |
| Mouse CXCL2/MIP-2 Quantikine | R&D | MM200 |
| ELISA kit | | |

Part B.: Human Epidermal Keratinocytes

Normal Human Epidermal Keratinocytes (NHEK) (PromoCell No. C-12008);

Rinse Buffer: Sterile D-PBS;

Complete Medium: Keratinocyte Growth Medium 2;

Controls:

Positive Control: 40 mJ/cm² UVB,

Negative Control: No UVB irradiation;

Test kits: (ELISA kit)

| Item | Brand | Cat. No |
|---|---|---|
| Human IL-6 Quantikine ELISA kit | R&D | M6000B |
| Human IL-8 Quantikine ELISA kit | R&D | MM200 |

4.2. General Procedure:

Procedure A. Raw264.7:

a. Rinse the confluent Raw264.7 cells with sterile D-PBS then trypsinize all cells and perform cell counting.

b. Dilute the cell density to 2E+05/mL and seed 0.1 mL cell liquid (equals to 2E+04 cells) each well in 96-well plate.

c. Incubate Raw264.7 cells at 37° C., 5% CO₂ for about 24 hours.

d. Prepared 2× dosing solution of testing material in 2× Testing Medium (means w/ 200 ng/mL [symbol] LPS&20 ng/mL [symbol] IFN-r): 0.8%, 0.4%, 0.2%, 0.1% (Final testing material: 0.4%, 0.2%, 0.1%, 0.05%)

e. Do not remove the supernatant. Add 100 μL controls and testing materials in different concentrations into Raw264.7 cells in 96 well plates. Controls/Test materials should be tested at least three wells.

f. Incubate Raw264.7 cells at 37° C., 5% CO₂ for about 24 hours.

g. After incubating for about 24 hours, collect the supernatant of controls and testing materials.

h. Determine the IL-6/MIP-2 content of the supernatant by R&D ELISA kit as mentioned previously.

Procedure B. NHEK:

a. Rinse the confluent NHEK cells with sterile D-PBS then trypsinize all cells and perform cell counting.

b. Dilute the cell density to 4˜8E+04/mL and seed 0.5 mL cell liquid (equals to 2˜4E+04 cells) each well in 24 well plate.

c. Incubate NHEK cells at 37° C., 5% CO₂ until subconfluence.

d. Prepared dosing solution of testing material in Complete Medium: 0.4%, 0.2%, 0.1%, 0.05%.

e. Rinse the NHEK cells with sterile D-PBS and discard rinse buffer. Add 400 μL of controls and testing materials in different concentrations into NHEK cells in 24 well plates.

f. Pre-incubate NHEK cells with testing material at 37° C., 5% CO₂ for about 6 hours.

g. After pre-incubating for about 6 hours, wash the NHEK cells with sterile D-PBS for twice then add 500 sterile D-PBS for UVB irradiation.

h. UVB intensity is about 2300 μW/cm² and UVB energy is about 40 mJ/cm².

i. After UVB irradiation, discard sterile D-PBS and wash again. Add 400 μL of the same dosing solution in to each wells.

j. Post-incubate at 37° C., 5% CO₂ for about 18 hours.

k. After post-incubating for 18 hours, collect the supernatant of controls and testing materials.

l. Determine the IL-6/IL-8 content of the supernatant by R&D ELISA kit as mentioned previously.

4.3. Data Analysis:

$$Percentage to positive control ( \% ) = \frac{\begin{matrix}IL \text{-} 6 / IL \text{-} 8 ( MIP / 2 ) content of negative control \\ or testing materials\end{matrix}}{IL \text{-} 6 / IL \text{-} 8 ( MIP / 2 ) content of Positive control} \times 100 \%$$

The supernatant of controls and testing materials.

4.4. Cell Viability is Tested by Neutral Red Uptake Experience or MTT Assay.

The following Table 3 lists Mouse IL-6 production testing results of Ac-Gly-β-Ala treating Raw264.7 cells for 3-repeated experiments according to the above mentioned experiments. As shown in Table 3, 0.4% Ac-Gly-β-Ala can inhibit IL-6 production to 34% comparing to positive control.

TABLE 3
Data Analysis of Mouse IL-6 Content inhibited by Ac-Gly-β-Ala

| | | | Entry | | | | |
|---|---|---|---|---|---|---|---|
| | | | Entry 1 | Entry 2 | Entry 3 | Aver- | |
| Passage number | | | N + 7 | N + 3 | N + 6 | age | Stdev |
| Negative Control | | | 0% | 0% | 0% | 0% | 0% |
| LPS & | Positive | | 100% | 100% | 100% | 100% | 0% |
| IFN-r | Control | | | | | | |
| | Ac-Gly- | 0.40% | 24% | 40% | 40% | 34% | 9% |
| | β-Ala | 0.20% | 64% | 58% | 42% | 54% | 11% |
| | | 0.10% | 85% | 96% | 78% | 86% | 9% |
| | | 0.05% | 91% | 105% | 95% | 97% | 8% |

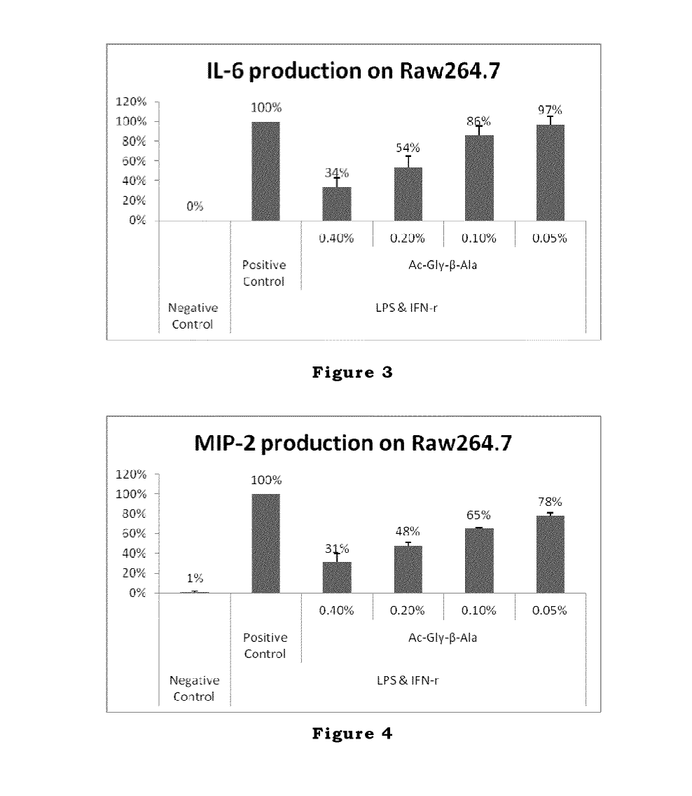

FIG. 3 shows 0.05%-0.4% of Ac-Gly-β-Ala dose-dependently inhibited IL-6 production of the Table 3. From FIG. 3, the dose-dependent trend is obvious.

Mouse MIP-2 production testing results of Ac-Gly-β-Ala treating Raw264.7 cells for 3-repeated experiments are listed in Table 4. As shown in Table 4, 0.4% Ac-Gly-β-Ala can inhibit MIP-2 production to 31% comparing to positive control. And FIG. 4 shows 0.05%-0.4% of Ac-Gly-β-Ala inhibited MIP-2 production in dose-dependent manner.

TABLE 4
Data Analysis of Mouse MIP-2 Content inhibited by Ac-Gly-β-Ala

| | | | Entry | | | | |
|---|---|---|---|---|---|---|---|
| | | | Entry 1 | Entry 2 | Entry 3 | Aver- | |
| Passage number | | | N + 5 | N + 3 | N + 6 | age | Stdev |
| Negative Control | | | 0% | 1% | 1% | 1% | 1% |
| LPS & | Positive | | 100% | 100% | 100% | 100% | 0% |
| IFN-r | Control | | | | | | |
| | Ac-Gly- | 0.40% | 21% | 32% | 38% | 31% | 9% |
| | β-Ala | 0.20% | 50% | 44% | 48% | 48% | 3% |
| | | 0.10% | 64% | 66% | 65% | 65% | 1% |
| | | 0.05% | 75% | 79% | 81% | 78% | 3% |

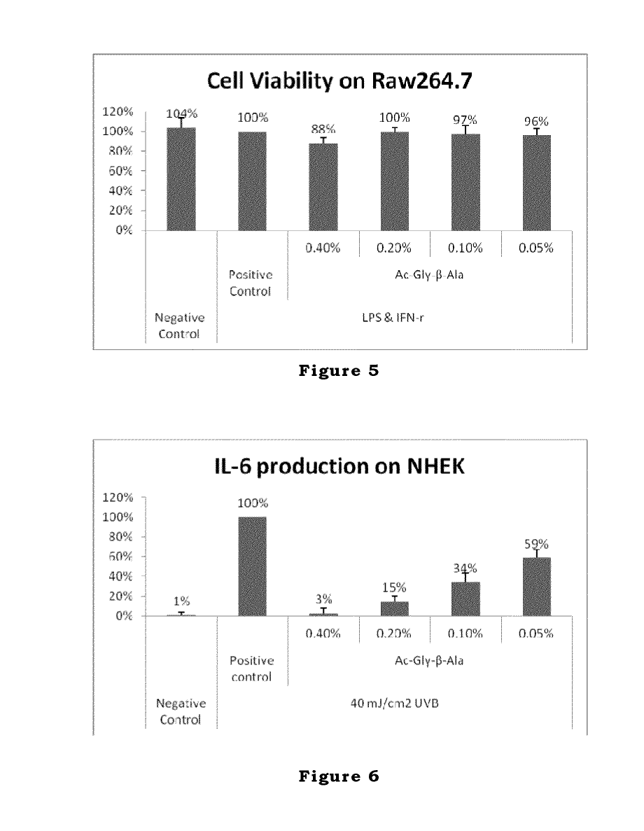

Cell viability testing results of Ac-Gly-β-Ala treating Raw264.7 cells for 3-repeated experiments are listed in Table 5. Cell viability is higher than 85% in all of dosing solution. FIG. 5 presents 0.05˜0.4% Ac-Gly-β-Ala shows good cell viability on Raw264.7.

TABLE 5
Data Analysis of cell viability on Raw264.7 by Ac-Gly-β-Ala

| | | | Entry | | | | |
|---|---|---|---|---|---|---|---|
| | | | Entry 1 | Entry 2 | Entry 3 | Aver- | |
| Passage number | | | N + 7 | N + 5 | N + 6 | age | Stdev |
| Negative Control | | | 89% | 109% | 104% | 104% | 10% |
| LPS & | Positive | | 100% | 100% | 100% | 100% | 0% |
| IFN-r | Control | | | | | | |
| | Ac-Gly- | 0.40% | 86% | 77% | 89% | 88% | 6% |
| | β-Ala | 0.20% | 90% | 98% | 91% | 100% | 4% |
| | | 0.10% | 95% | 104% | 87% | 97% | 9% |
| | | 0.05% | 101% | 101% | 89% | 96% | 7% |

5.4 Human IL-6 production testing results of Ac-Gly-β-Ala treating NHEK cells for 4-repeated experiments are listed in Table 6. Referred to Table 6, 0.4% Ac-Gly-β-Ala can inhibit IL-6 production to about 3% comparing to positive control. And FIG. 6 shows 0.05%-0.4% of Ac-Gly-β-Ala dose-dependently inhibited IL-6 production of Table 6.

TABLE 6
Data Analysis of Human IL-6 Content inhibited by Ac-Gly-β-Ala

| | | | Entry | | | | | |
|---|---|---|---|---|---|---|---|---|
| | | | Entry 1 | Entry 2 | Entry 3 | Entry4 | Aver- | |
| Passage number | | | 2 + 2 | 2 + 2 | 2 + 3 | 2 + 3 | age | Stdev |
| Negative Control | | | 0% | 0% | 0% | 5% | 1% | 3% |
| 40 | Positive | | 100% | 100% | 100% | 100% | 100% | 0% |
| mJ/cm² | Control | | | | | | | |
| UVB | Ac-Gly- | 0.40% | 0% | 0% | 0% | 10% | 3% | 5% |
| | β-Ala | 0.20% | 19% | 10% | 10% | 20% | 15% | 5% |
| | | 0.10% | 43% | 25% | 28% | 42% | 34% | 9% |
| | | 0.05% | 68% | 58% | 49% | 62% | 59% | 8% |

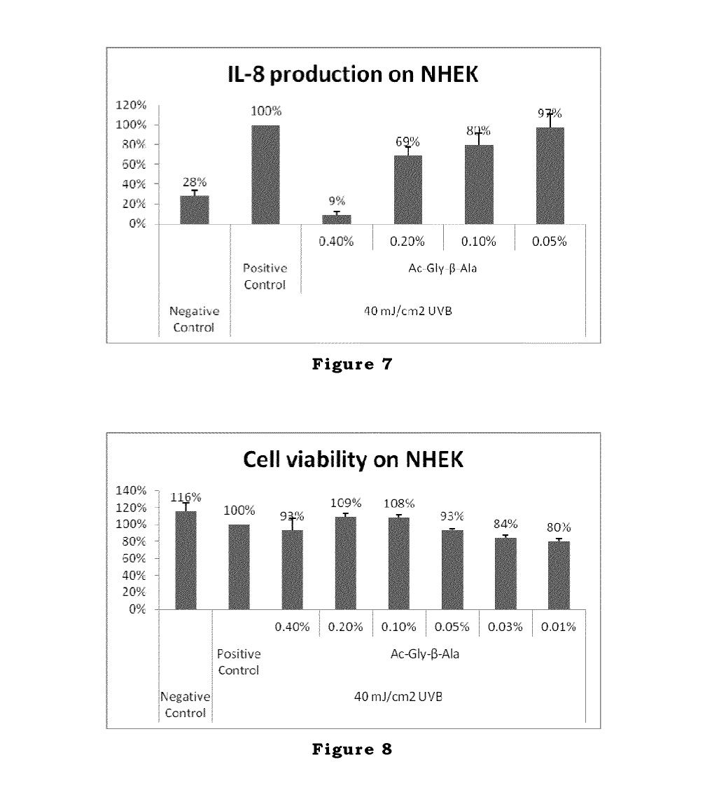

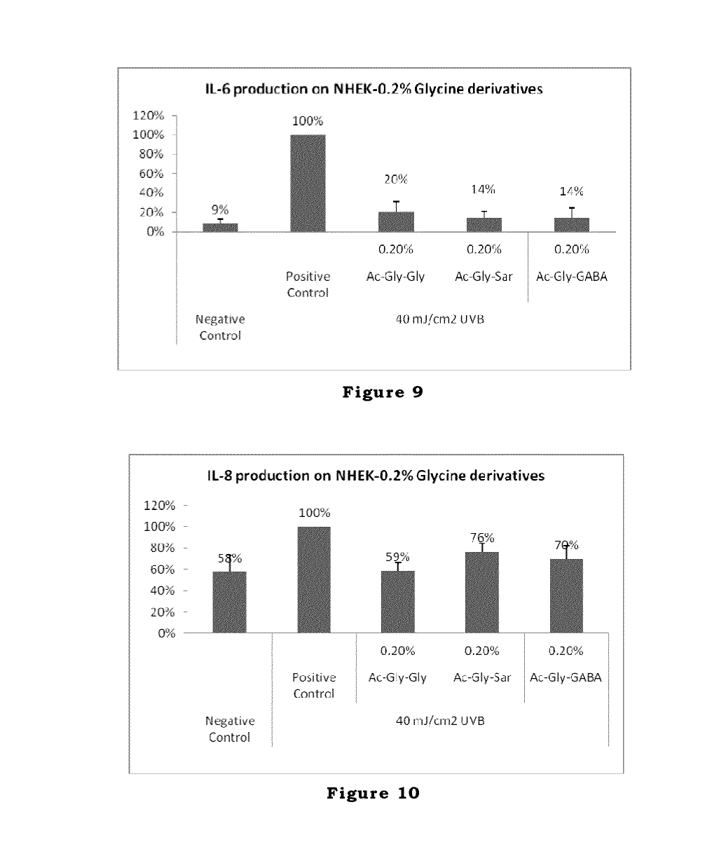

Human IL-8 production testing results of Ac-Gly-β-Ala treating NHEK cells for 3-repeated experiments are listed in Table 7. 0.4% Ac-Gly-β-Ala can inhibit IL-8 production to about 9% comparing to positive control. And FIG. 7 shows 0.05%—0.4% of Ac-Gly-β-Ala inhibited IL-8 production in dose-dependent manner.

TABLE 7
Data Analysis of Human IL-8 Content inhibited by Ac-Gly-β-Ala

| | | | Entry | | | | |
|---|---|---|---|---|---|---|---|
| | | | Entry 1 | Entry 2 | Entry 3 | Aver- | |
| Passage number | | | 2 + 2 | 2 + 3 | 2 + 3 | age | Stdev |
| Negtive Control | | | 24% | 35% | 24% | 28% | 6% |
| 40 | Positive | | 100% | 100% | 100% | 100% | 0% |
| mJ/cm² | Control | | | | | | |
| UVB | Ac-Gly- | 0.40% | 10% | 5% | 13% | 9% | 4% |
| | β-Ala | 0.20% | 77% | 72% | 59% | 69% | 9% |
| | | 0.10% | 76% | 70% | 93% | 80% | 12% |
| | | 0.05% | 107% | 81% | 103% | 97% | 14% |

Cell viability testing results of Ac-Gly-β-Ala treating NHEK cells for 3-repeated experiments are listed in Table 8, and shown as FIG. 8. Cell viability is higher than 80% in all of dosing solution.

TABLE 8
Data Analysis of cell viability on NHEK by Ac-Gly-β-Ala

| | | | Entry | | | | |
|---|---|---|---|---|---|---|---|
| | | | Entry 1 | Entry 2 | Entry 3 | Aver- | |
| Passage Number | | | 2 + 2 | 2 + 3 | 2 + 3 | age | Stdev |
| Negtive Control | | | 119% | 124% | 106% | 116% | 10% |
| 40 | Positive | | 100% | 100% | 100% | 100% | 0% |
| mJ/cm² | Control | | | | | | |
| UVB | Ac-Gly- | 0.40% | 106% | 95% | 79% | 93% | 14% |
| | β-Ala | 0.20% | 106% | 107% | 113% | 109% | 4% |
| | | 0.10% | 109% | 103% | 110% | 108% | 4% |
| | | 0.05% | 93% | 90% | 95% | 93% | 2% |
| | | 0.03% | 88% | 80% | 83% | 84% | 4% |
| | | 0.01% | 80% | 82% | 76% | 80% | 3% |

### Example 5

### Anti-Inflammatory Effect of Other Glycine Derivatives

We have reported the anti-inflammation of Ac-Gly-β-Ala as Example 4. In order to realize the anti-inflammatory effect of other glycine derivatives, in this example, we also try to treat normal human epidermal kerationytes by other glycine derivatives: (2-acetylamino-acetylamino)-acetic acid (Acetyl-Glycine-Glycine; Ac-Gly-Gly), [(2-Acetylamino-acetyl)-methyl-amino]-acetic acid (Acetyl-Glycine-Sarcosine; Ac-Gly-Sar), and 4-(2-Acetylamino-acetylamino)-butyric acid (Acetyl-Glycine-γ-aminobutyric acid; Ac-Gly-GABA). The formula of the mentioned Ac-Gly-Gly, Ac-Gly-Sar, and Ac-Gly-GABA are as the following.

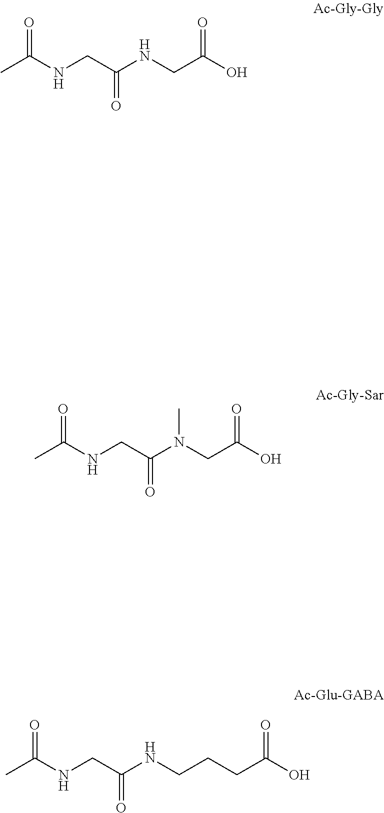

Normal Human Epidermal Keratinocytes (NHEK) were treated by the above-mentioned glycine derivatives for evaluating their anti-inflammatory effect on keratinocytes in this example.

Material and General Procedure

As same as Example 4

TABLE 9
Data Analysis of Human IL-6 Content
inhibited by Glycine derivatives

| | | | Entry | | | | |
|---|---|---|---|---|---|---|---|
| | | | Entry 1 | Entry 2 | Entry 3 | Aver- | |
| Passage number | | | 2 + 2 | 2 + 3 | 2 + 3 | age | Stdev |
| Negtive Control | | | 24% | 35% | 24% | 28% | 6% |
| 40 | Positive | | 100% | 100% | 100% | 100% | 0% |
| mJ/cm² | Control | | | | | | |
| UVB | Ac-Gly- | 0.20% | 12% | 16% | 33% | 20% | 11% |
| | Gly | | | | | | |
| | Ac-Gly- | 0.20% | 14% | 21% | 7% | 14% | 7% |
| | Sar | | | | | | |
| | Ac-Gly- | 0.20% | 11% | 25% | 6% | 14% | 10% |
| | GABA | | | | | | |

TABLE 1O
Data Analysis of Human IL-8 Content inhibited by Glycine derivatives

| | | | Entry | | | | | |
|---|---|---|---|---|---|---|---|---|
| | | | Entry 1 | Entry 2 | Entry 3 | Entry 4 | Aver- | |
| Passage number | | | 2 + 3 | 2 + 4 | 2 + 5 | 2 + 6 | age | Stdev |
| Negtive Control | | | 49% | 43% | 80% | 58% | 58% | 16% |
| 40 | Positive | | 49% | 43% | 80% | 58% | 58% | 16% |
| mJ/cm2 | Control | | | | | | | |
| UVB | Ac-Gly- | 0.20% | — | 68% | 54% | 53% | 59% | 8% |
| | Gly | | | | | | | |
| | Ac-Gly- | 0.20% | 73% | 70% | 74% | 88% | 76% | 8% |
| | Sar | | | | | | | |
| | Ac-Gly- | 0.20% | 59% | 70% | 64% | 87% | 70% | 12% |
| | GABA | | | | | | | |

The results show that glycine derivatives with the structure of this invention have the potential inhibitory effect on inflammation.

Another preferred embodiment according to this specification discloses an anti-inflammatory composition, comprising a glycine derivatives with addition amount of 0.05-10 wt % (weight ratio) of the anti-inflammatory composition. The glycine derivatives has a function of inhibiting inflammation. The general formula of the mentioned glycine derivatives is as the following.

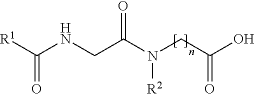

In the above-mentioned general formula, R¹ represents a C₁-C₄ alkyl group, R² represents a hydrogen atom or a methyl group, and n represents an integer of 1-6.

The mentioned anti-inflammatory composition can be applied in skin care preparations, for example, facial and/or body cleansers, toner, moisturizing spray, face masks, serum, day cream, night cream, eye cream, feminine halo cream, body cream, hand cream, hand wash, body wash, feminine hygiene cleanser. Preferably, the anti-inflammatory composition of this invention can be applied in topical cosmetic or pharmaceutical compositions as skin care preparations, or functional preparations.

Another preferred embodiment according to this specification discloses a composition for reducing melanophilin expression comprising a glycine derivatives with addition amount of 0.05-10 wt % (weight ratio) of the composition for reducing melanophilin expression. The glycine derivatives has a function of reducing melanophilin expression. According to one preferred example of this embodiment, the composition can efficiently reduce melanophilin expression and then attenuate melanosome transport, so that the pigmentation could be inhibited. The general formula of the mentioned glycine derivatives is as the following.

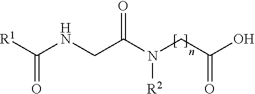

In the above-mentioned general formula, R¹ represents a C₁-C₄ alkyl group, R² represents a hydrogen atom or a methyl group, and n represents an integer of 1-6.

The mentioned composition for reducing melanophilin expression can be applied in skin care preparations, for example, facial and/or body cleansers, toner, moisturizing spray, face masks, serum, day cream, night cream, eye cream, feminine halo cream, body cream, hand cream, hand wash, body wash, feminine hygiene cleanser. Preferably, the composition for reducing melanophilin expression of this invention can be applied in topical cosmetic or pharmaceutical compositions as skin care preparations, or functional preparations.

The cosmetic compositions in accordance with the invention can be in the form of a liquid, lotion, a thickened lotion, a gel, a cream, a milk, an ointment, a paste, a powder, a make-up, or a solid tube stick and can be optionally packaged as an aerosol and can be provided in the form of a mousse such as a aerosol mousse, a foam or a spray foams, sprays, sticks, a gel, a plaster, a powder, a cleanser, a soap or aerosols or wipes. Preferred topical compositions comprise a cream, a gel, an ointment, a lotion a tincture, a spray, a mousse, a cleansing composition or foam. The following examples are concerned with topical compositions which may be prepared by procedures known per se in the art.

### Example 6

### Facial Cleanser

| part | Ingredient | wt % |
|---|---|---|
| A | Water | To 100 |
| A | Ac-Gly-β-Ala | 0.2 |
| B | Sodium Lauroyl Glutamate | 21 |
| B | TEA Lauroyl Glutamate | 1 |
| B | Laureth-9 | 1 |
| B | Sodium Cocoyl Isethionate | 17 |
| C | PEG-80 Glyceryl Cocoate | 1.8 |
| C | Polyethylene glycol | 4 |
| C | Cetearyl Alcohol | 1 |
| C | Glyceryl Stearate | 1 |
| C | Glycol Distearate | 3.5 |
| C | Talc | 0.5 |
| D | Phytosteryl Hydroxystearate | 0.4 |
| D | Castoryl Maleate | 0.5 |

Appearance: White Paste;

pH Value (10% Solution/25° C.): 5.70;

Procedure:
1. Ac-Gly-β-Ala dissolves in water.
2. Add Part B into Part A and heat them to 80-85° C. until completely dissolved.
3. Add Part C into Part A/B until completely transparent and ensure no granular.
4. Cooling to 60° C. with slowly stirring.
5. Add Part D into batch and blend well.

### Example 7

### Whitening Serum

| part | Ingredient | wt % |
|---|---|---|
| A | Water | To 100.00 |
| A | Xanthan Gum | 0.05 |
| B | Hydroxyethylcellulose | 20 |
| B | Aloe Barbadensis Leaf Juice | 1 |
| B | PEG-120 Jojoba Esters | 0.6 |
| B | Butylene Glycol | 3 |
| B | Methylisothiazolinone | 0.1 |
| C | Glycerin | 3 |
| C | Hydrolyzed Jojoba Esters (and) Water | 1 |
| C | Octoxynol-11 (and) Polysorbate 20 | 1.5 |
| D | Ac-Gly-β-Ala | 1 |
| D | Sodium Hydroxide (10%) | 0.54 |
| D | Water | 10 |

Appearance: Transparent Flow Gel;

pH Value (25° C.): 4.10;

Viscosity (S63/60 rpm/30 Sec/25° C.): 182 cPs;

Procedure:
1. Add Xanthan Gum into water and mix until uniform.
2. Add Part B ingredients in sequence and mix well.
3. Pre-mix Part C and Part D separately.
4. Add Part C and Part D into Part A/B with stirring.

### Example 8

### Whitening Spot Corrector

| part | Ingredient | wt % |
|---|---|---|
| A | Cetyl Alcohol (and) Glyceryl Stearate | 6.5 |
| | (and) PEG-75 Stearate (and) Ceteth-20 | |
| | (and) Steareth-20 | |
| A | Cetearyl Alcohol | 2 |
| A | Isohexadecane | 7 |
| A | Octyldodecyl Myristate | 5 |
| A | Macadamia Integrifolia Seed Oil | 3 |
| A | Octyldodecyl Stearoyl Stearate | 2 |
| A | Avocado (Persea Gratissima) Oil | 2 |
| A | Phytosteryl Hydroxystearate | 0.5 |
| A | Dimethicone | 3 |
| B | Water | To 100 |
| B | Xanthan Gum | 0.12 |
| C | Disodium EDTA | 0.05 |
| C | Allantoin | 0.1 |
| C | Dipotassium Glycyrrhizinate | 0.15 |
| C | Glycerin | 1.5 |
| C | Butylene Glycol | 3.5 |
| D | Water | 12 |
| D | Ac-Gly-β-Ala | 2 |
| D | Sodium Hydroxide | 1.05 |
| E | Ethoxydiglycol | 0.2 |
| E | Salicylic Acid | 0.1 |
| E | Sodium Hydroxide | 0.12 |
| F | Silica | 0.5 |
| F | Fragrance | 0.03 |
| F | Phenoxyethanol (and) Methylparaben | 0.8 |
| | (and) Ethylparaben (and) Propylparaben | |

Appearance: White Cream;

pH Value (25° C.): 4.10;

Viscosity (S64/3 rpm/30 Sec/25° C.): 80,383 cP;

Procedure:
1. Add Part C into Part B and mix well.
2. Part B/C heat up till 75° C., then add Part D into Part B/C and mix well.
3. Heat Part A to 75° C. Add Part B/C/D into Part A.
4. Cool down to 40° C., add Part E and Part F into the batch and blend well.

### Whitening Spot Corrector

| | |
|---|---|
| Batch No. | 20130109 |
| Formulation date | 2013/01/09 |
| Expected weight | 1500 g |
| Additional water percentage | 3% |
| Final weight | 1472.27 g |

### Formulation Stability Assessment

| | | 90 Days | | |
|---|---|---|---|---|
| | 3 Days | 5° C. | 25° C. | 45° C. |
| Appearance(25° C.) | White | White | White | White |
| | Cream | Cream | Cream | Cream |
| ¹pH Value(25° C.) | 4.10 | 4.01 | 4.16 | 4.18 |
| ²Viscosity (cPs) | 80,383 cP | 69,185 cP | 67,186 cP | 74,784 cP |
| S64/3 rpm/30 Sec/25° C. | | | | |
| Evaluation of stability | Pass | Pass | Pass | Pass |

### Example 9

### Toner

| part | Ingredient | % |
|---|---|---|
| A | Water | To 100 |
| A | Dipotassium Glycyrrhizinate | 0.15 |
| A | Disodium EDTA | 0.05 |
| A | Allantoin | 0.1 |
| A | PEG-120 Jojoba Esters | 0.6 |
| A | Glycerin | 2 |
| A | Butylene Glycol | 2 |
| A | Propylene Glycol | 4 |
| A | Hydroxyethylcellulose (2%) | 5 |
| A | Sodium Hyaluronate (1%) | 2 |
| A | Methylisothiazolinone | 0.08 |
| A | Phenoxyethanol | 0.5 |
| A | Sodium Hydroxide (10%) | 0.01 |
| B | Fragrance | 0.01 |
| B | Polyoxyethylenated Alkylphenol and | 0.03 |
| | Polyoxyethylenated Sorbitan | |
| | Monolaurate | |
| C | Water | 3 |
| C | Ac-Gly-β-Ala | 0.2 |

Appearance: Transparent Liquid;

pH Value (25° C.): 4.04;

Procedure:
1. Pre-mix Part A, Part B, and Part C separately.
2. Add Part B and Part C into Part A and mix well.

### Example 10

### Whitening Lotion

| part | Ingredient | % |
|---|---|---|
| A | Cetyl Alcohol (and) Glyceryl Stearate | 4 |
| | (and) PEG-75 Stearate (and) Ceteth-20 | |
| | (and) Steareth-20 | |
| A | Cetearyl Alcohol | 1 |
| A | Macadamia Integrifolia Seed Oil | 2 |
| A | Jojoba Esters | 1.5 |
| A | Jojoba Esters | 2 |
| A | Phytosteryl Hydroxystearate | 0.5 |
| A | Cyclopentasiloxane | 2 |
| B | Water | To 100 |
| B | Disodium EDTA | 0.05 |
| B | Allantoin | 0.1 |
| B | Dipotassium Glycyrrhizinate | 0.15 |
| B | PEG-120 Jojoba Esters | 3 |
| B | Carbomer (2%) | 30 |
| C | Water | 3 |
| C | Ac-Gly-β-Ala | 0.5 |
| C | Sodium Hydroxide (10%) | 0.25 |
| D | Sodium Hydroxide (10%) | 0.9 |
| D | Pentaerythrityl tetraisostearate (and) | 2 |
| | Mineral oil (and) Disteardimonium | |
| | hectorite (and) Propylene carbonate (and) | |
| | Palmitoyl oligopeptide | |
| D | Pentaerythrityl tetraisostearate (and) | 3 |
| | Mineral oil (and) Disteardimonium | |
| | Hectorite (and) Propylene Carbonate | |
| | (and) Palmitoyl Hexapeptide-12 | |
| E | Propylene Glycol (and) Water (and) Horse | 1 |
| | Chestnut Extract | |
| E | Aloe Barbadensis Leaf Juice | 1 |
| F | Phenoxyethanol | 0.5 |
| F | Potassium Sorbate | 0.3 |

Appearance: White Lotion;

pH Value (25° C.): 4.57;

Procedure:
1. Pre-mix Part B and Part C. Add Part C into Part B and mix well.
2. Part A and Part B/C heat up till 75° C. separately, then add Part B/C into Part A and mix well.
3. Add Part D ingredient in sequence into batch and blend well.
4. Cool down to 40° C., add Part E and Part F into the batch with stirring.

### Example 11

### Sunscreen

| part | Ingredient | wt % |
|---|---|---|
| A | Ethylhexyl Methoxycinnamate | 5 |
| A | Bis-Ethylhexyloxyphenol Methoxyphenyl Triazine | 1.7 |
| B | Octocrylene | 10 |
| B | C12-14 Alkyl Benzoate | 7 |
| B | Glyceryl Dibehenate (and) Tribehenin (and) | 1 |
| | Glyceryl Behenate | |
| B | Polyglycerol-3 Beeswax | 1.8 |
| B | PEG-30 dipolyhydroxystearate | 3.5 |
| B | Microstalline Wax | 0.5 |
| B | C₁₃₋₄₅ Alkyl Methicone (and) C₃₀₋₄₅ Olefin | 1 |
| C | Titanium Dioxide (And) Caprylic/Capric | 12.8 |
| | Triglyceride (And) Alumina (And) Silica (And) | |
| | Polyhydroxystearic Acid | |
| D | Water | To 100 |
| D | Ac-Gly-β-Ala | 0.3 |
| D | Sodium Hydroxide(10%) | 0.15 |
| D | Glycerin | 3 |
| D | Sclerotium Gum | 2 |
| D | Magnesium Sulfate | 1 |
| D | Disodium EDTA | 0.2 |
| D | Polysorbate 80 | 0.25 |
| E | Methylene Bis-benzotriazolyl | 8 |
| | Tetramethylbutylphenol (and) Aqua (and) Decyl | |
| | Polyglucoside (and) Propylene Glycol (and) Xanthan | |
| | Gum | |
| F | Cyclopentasiloxane (and) Cyclohexasiloxane | 10 |
| F | Isopropylparaben (and) Isobutylparaben (and) | 0.15 |
| | Butylparaben | |

Procedure:
1. Heat Part A until dissolved, then add Part B into Part A and heat Part A/B to 80° C.
2. Add Part C into Part A/B and mix well.
3. Pre-mix Part D and Heat Part D to 80° C.
4. Keep the temperature, homogenize Part A/B/C at 11000 rpm using a homogenizer.
5. Add Part D into batch and mix under homogenizer until the batch is smooth and uniform.
6. Remove to stirrer and cools to 40° C., add Part E and Part F into batch and blend well.

### Example 12

### Whitening BB Cream

| part | Ingredient | % |
|---|---|---|
| A | Titanium Dioxide (And) Triethoxy Caprylylsilane | 6.3 |
| A | Iron Oxides (C.I. 77492) (and) Triethoxy | 0.77 |
| | Caprylylsilane | |
| A | Iron Oxides (C.I. 77491) (and) Triethoxy | 0.08 |
| | Caprylylsilane | |
| A | Iron Oxides (C.I. 77499) (and) Triethoxy | 0.08 |
| | Caprylylsilane | |
| A | Diisostearyl Malate | 3.2 |
| B | Ethylhexyl Methoxycinnamate | 2 |
| B | Isohexadecane | 4 |
| B | Cyclopentasiloxane | 8 |
| B | Phenoxyethanol | 0.5 |
| B | Cetyl PEG/PPG-10 Dimethicone | 2 |
| B | Fragrance | 0.1 |
| C | Water | To 100 |
| C | Ac-Gly-β-Ala | 0.5 |
| C | Sodium Hydroxide(10%) | 0.25 |
| C | Butylene Glycol | 3 |
| C | Glycerin | 2 |
| C | Sodium Chloride | 0.5 |
| C | Potassium Sorbate | 0.3 |
| C | Disteardimonium Hectorite (and) Propylene | 2 |
| | Carbonate (and) Palmitoyl hexapeptide-12 | |

Appearance: Slightly Yellowish Cream;

pH value of water phase (25° C.);

Viscosity (S64/30 rpm/25° C./30 Sec/D): cPs;

Procedure:
1. Pre-mix Part A until uniform.
2. Add Part B into Part A and blend well.
3. Pre-mix Part C.
4. Slowly add Part C into Part A/B drop by drop with stirring.

In summary, this application has reported method and composition for inhibiting inflammation and reducing melanophilin expression with glycine derivatives, and the composition thereof. According to this invention, the mentioned glycine derivatives can efficiently inhibit skin inflammation. The glycine derivatives also can present significant melanophilin expression reduction, so that the glycine derivatives of this invention can provide skin lightening performance through inhibiting melanin transportation. This mentioned composition of this invention comprises the glycine derivatives, and shows melanophilin expression reduction and anti-inflammatory effect. The mentioned composition can be applied in topical cosmetic or pharmaceutical compositions as skin care preparations, or functional preparations.

Obviously many modifications and variations are possible in light of the above teachings. It is therefore to be understood that within the scope of the appended claims the present invention can be practiced otherwise than as specifically described herein. Although specific embodiments have been illustrated and described herein, it is obvious to those skilled in the art that many modifications of the present invention may be made without departing from what is intended to be limited solely by the appended claims.

What is claimed is:

1. A method for reducing melanophilin expression, comprising:
applying glycine derivatives to a target region, wherein the glycine derivatives has a general structure shown as the following: 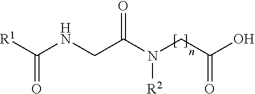
wherein R¹ represents a C1-C4 alkyl group, R² represents a hydrogen atom or methyl group, and n represents an integer of 1-6.

2. The method according to claim 1, wherein the glycine derivatives is 3(2-acetylamino-acetylamino)-propionic acid having a structure shown in the following:

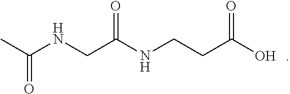

3. The method according to claim 1, wherein the glycine derivatives is 4-(2-Acetylamino-acetylamino)-butyric acid having a structure shown in the following:

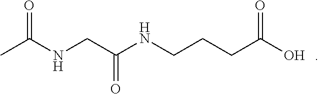

4. The method according to claim 1, wherein the glycine derivatives is [(2-Acetylamino-acetyl)-methyl-amino]acetic acid having a structure shown in the following:

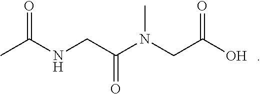

5. The method according to claim 1, wherein the target region is selected from one of the following: cells, skin of a mammal.

6. A composition for reducing melanophilin expression, comprising:
a glycine derivatives with addition amount of 0.05˜10 wt % (weight ratio) of the reducing melanophilin expression composition, wherein said glycine derivative has a function of reducing melanophilin expression and has a structure shown as the following: 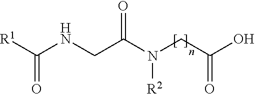
wherein R¹ represents a C1-C4 alkyl group, R² represents a hydrogen atom or methyl group, and n represents an integer of 1-6.

7. The composition according to claim 6, wherein the glycine derivatives is 3(2-acetylamino-acetylamino)-propionic acid having a structure shown in the following:

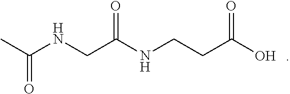

8. The composition according to claim 6, wherein the glycine derivatives is 4-(2-Acetylamino-acetylamino)-butyric acid having a structure shown in the following:

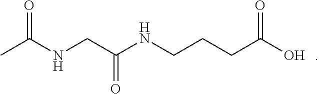

9. The composition according to claim 6, wherein the glycine derivatives is [(2-Acetylamino-acetyl)-methyl-amino]-acetic acid having a structure shown in the following:

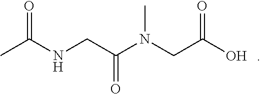

10. A anti-inflammatory composition, comprising:
a glycine derivatives with addition amount of 0.05˜10 wt % (weight ratio) of the whitening composition, wherein said glycine derivatives has a function of inhibiting inflammation and has a structure shown as the following: 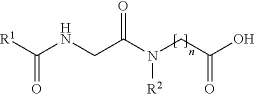
wherein R¹ represents a C1-C4 alkyl group, R² represents a hydrogen atom or a methyl group, and n represents an integer of 1-6.

11. The composition according to claim 10, wherein the glycine derivatives is 3(2-acetylamino-acetylamino)-propionic acid having a structure shown in the following:

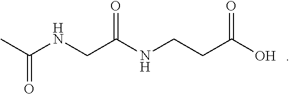

12. The composition according to claim 10, wherein the glycine derivatives is 4-(2-Acetylamino-acetylamino)-butyric acid having a structure shown in the following:

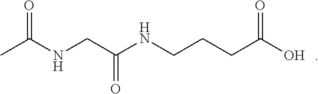

13. The composition according to claim 10, wherein the glycine derivatives is [(2-Acetylamino-acetyl)-methyl-amino]-acetic acid having a structure shown in the following:

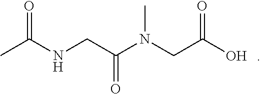

14. The composition according to claim 10, wherein the target region is selected from one of the following: cells, skin of a mammal.
